(19) United States
(12) Patent Application Publication
(10) Pub. No.: US 2019/0240074 A1
(43) Pub. Date: Aug. 8, 2019

(54) RELEASE PAPERS FOR WOUND TREAMENT DEVICES

(71) Applicant: Ethicon LLC, Guaynabo, PR (US)

(72) Inventor: Julian A. Quintero, Flemington, NJ (US)

(21) Appl. No.: 16/387,634

(22) Filed: Apr. 18, 2019

Related U.S. Application Data
(63) Continuation of application No. 29/648,487, filed on May 22, 2018.
Application No. 29/648,487 is a continuation of application No. 29/503,320, filed on Sep. 25, 2014, now Pat. No. D824525.

(51) Int. Cl. A61F 13/02

(52) U.S. Cl. CPC A61F 13/0259; A61F 13/0253; A61F 13/023

(57) ABSTRACT

The invention is related to wound treatment devices that comprise a three-part release liner assembly that permits accurate placement of the wound treatment device.

### CROSS-REFERENCE TO RELATED APPLICATIONS

This is a continuation application which claims priority to design application Ser. No. 29/648,487, filed May 22, 2018, which in turn claims priority to design application Ser. No. 29/503,320, filed Sep. 25, 2014, now U.S. D824,525 the disclosures of which are incorporated herein by reference.

### BACKGROUND OF THE INVENTION

This invention is concerned with wound treatment devices comprising a three-part release paper assembly.

### SUMMARY OF THE INVENTION

This invention is directed to a wound treatment device comprising a three-part release liner assembly detachably adhered to a wound treatment component, the release liner assembly comprises a first frame, a central section, and a second frame.

In one embodiment, the central section of the release liner assembly comprises a length and a width, wherein the width varies along the length of the central section and has a left edge and a right edge and wherein along the length of the central section, the width of the central section has a first width at the left edge which decreases to a second width, which further increases to a third width, which further decreases to a fourth width and which further increases to a fifth width at the right edge.

In another embodiment, the central section of the release liner assembly comprises a length and a width, wherein the width varies along the length of the central section and has a left edge and a right edge and wherein along the length of the central section, the width of the central section has a first width at the left edge that increases to a second width for a length and then decreases to a third width at the right edge.

In yet a further embodiment, the central section of the release liner assembly comprises a length and a width, wherein the central section and has a left edge and a right edge and wherein along the length of the central section, the width of the central section has a width at the left edge wherein the width not vary along the length of central section and is maintained up to the width at the right edge.

### BRIEF DESCRIPTION OF THE DRAWINGS

FIGS. 1-6 depict a wound treatment device comprising a three-part release liner in accordance with an embodiment of the invention.

FIGS. 7-12 depict a wound treatment device comprising a three-part release liner in accordance with an embodiment of the invention.

FIGS. 13-18 depict a wound treatment device comprising a three-part release liner in accordance with an embodiment of the invention.

FIGS. 19-24 depict a wound treatment device comprising a three-part release liner in accordance with an embodiment of the invention.

FIGS. 25-30 depict a wound treatment device comprising a three-part release liner in accordance with an embodiment of the invention.

FIGS. 31-36 depict a wound treatment device comprising a three-part release liner in accordance with an embodiment of the invention.

FIGS. 37-42 depict a wound treatment device comprising a three-part release liner in accordance with an embodiment of the invention.

### DETAILED DESCRIPTION OF PREFERRED EMBODIMENTS OF THE INVENTION

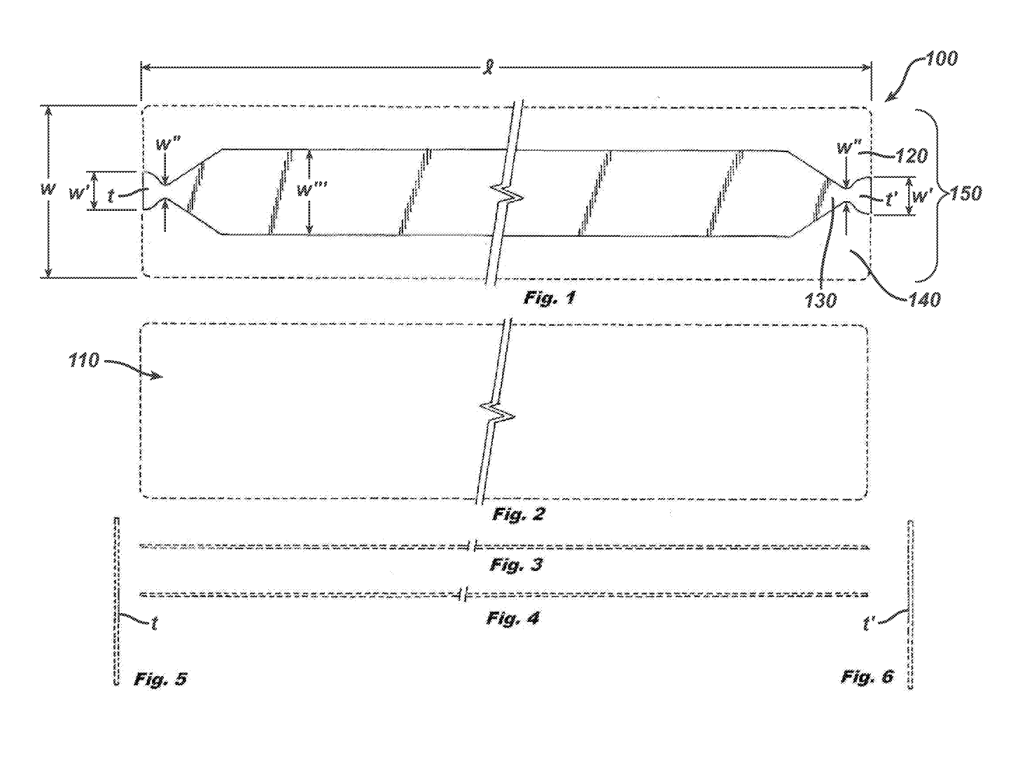

FIGS. 1-7 depict wound treatment device 100 having an overall length l and overall width w and comprising a three-part release liner assembly 150 in accordance with one embodiment of the invention. FIG. 1 is an underside view of device 100 comprising a release or backing assembly 150 comprising first frame 120, central section 130 having pull tabs t and t′, and second frame 140. Central section 130 has width w′ at the left edge of central section 130 which reduces to width w″ and expands to width w′″ before reducing to w″ and expanding to w′ at the right edge of central section 130.

FIG. 2 is a top view of device 100 which comprises wound treatment component 110. FIGS. 3 and 4 are top and bottom edge views of device 100, respectively. FIGS. 5 and are left-side and right-side views, respectively of device 100.

Many embodiments are evident for device 100 depending on the specific dimensions one skilled in the art may select and adapt to treat wounds or incisions of various sizes and shapes.

Device 100 may be of any length l and width w that permits the user to effectively apply to treat a wound.

Wound treatment component 110 may be of any length l and width w that permits the user to effectively treat a wound.

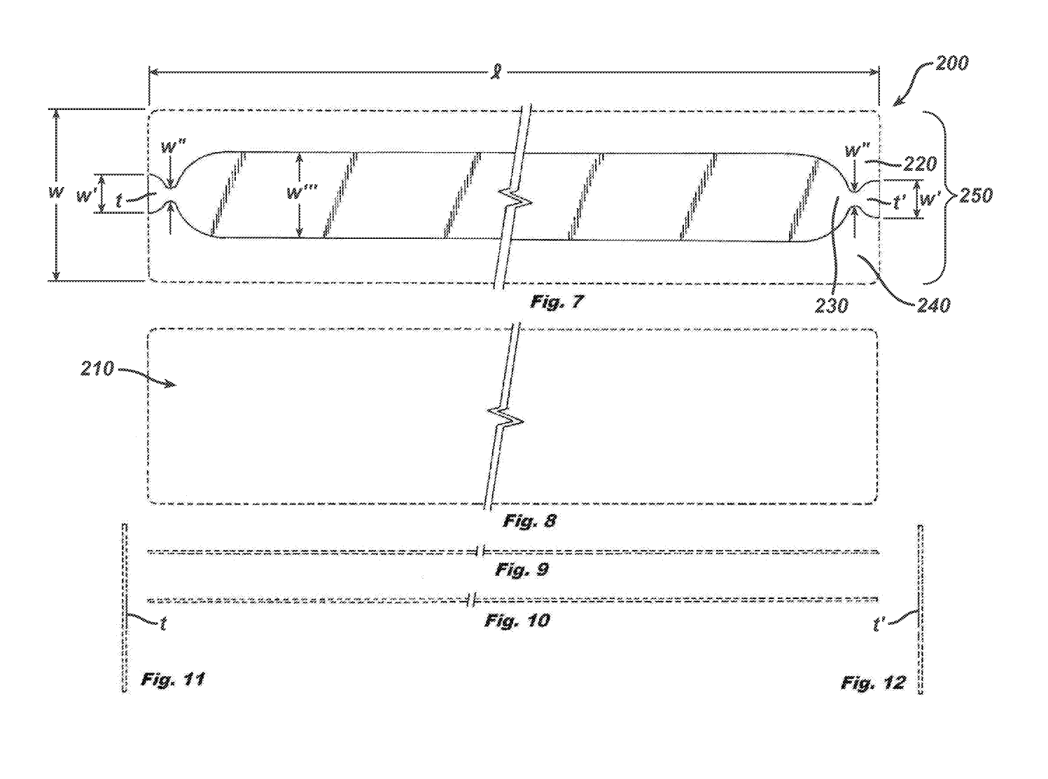

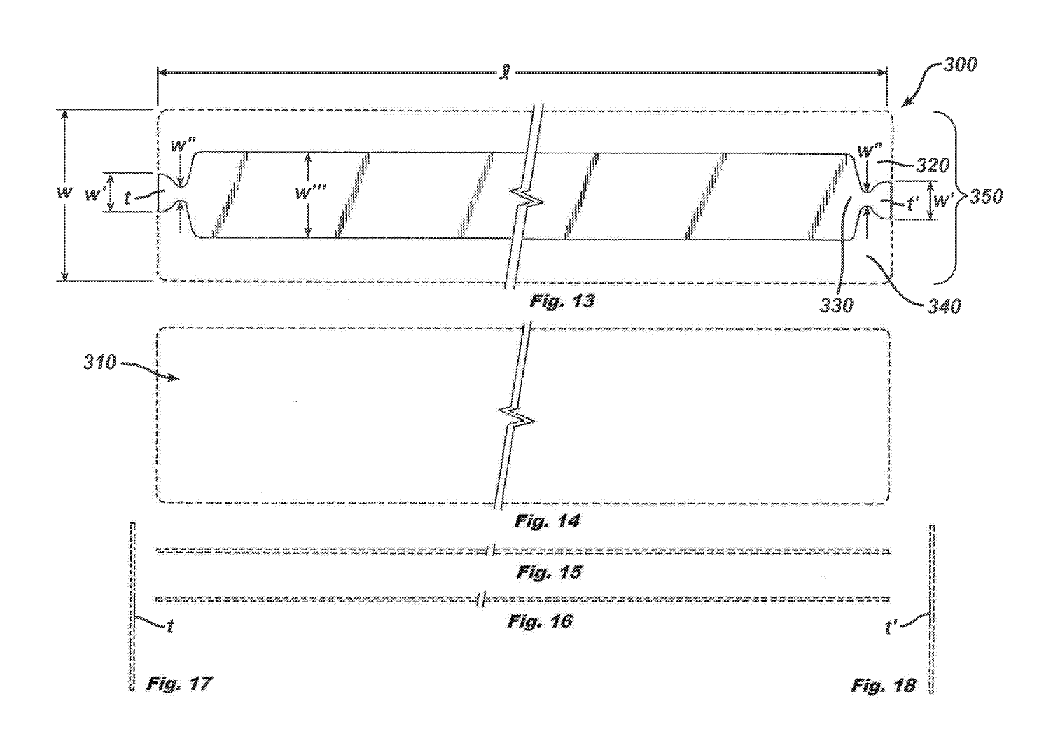

As a point of clarity, the device illustrations of FIGS. 7-12 to FIGS. 37-42, inclusive, depict variations in the design of central section 130 of FIGS. 1-6. As such, all previously defined parameters described for FIGS. 1-6 apply and have the same meaning. The numbering of the corresponding elements of the variously depicted embodiments for the devices follow the same numbering convention as in FIGS. 1-6 and only differ in that the numbering corresponds to series numbering specific to each set of illustrations (e.g., 200 series numbering for elements of FIGS. 7-12, 300 series numbering for elements of FIGS. 13-18, etc.) Therefore further detailed description is not provided as one skilled in the art would appreciate and understand the commonality and defined meaning of the elements and dimensions described therein.

FIGS. 7-12 depict a wound closure device comprising a three-part release liner in accordance with another embodiment of the invention.

FIGS. 13-18 depict a wound closure device comprising a three-part release liner in accordance with a further embodiment of the invention.

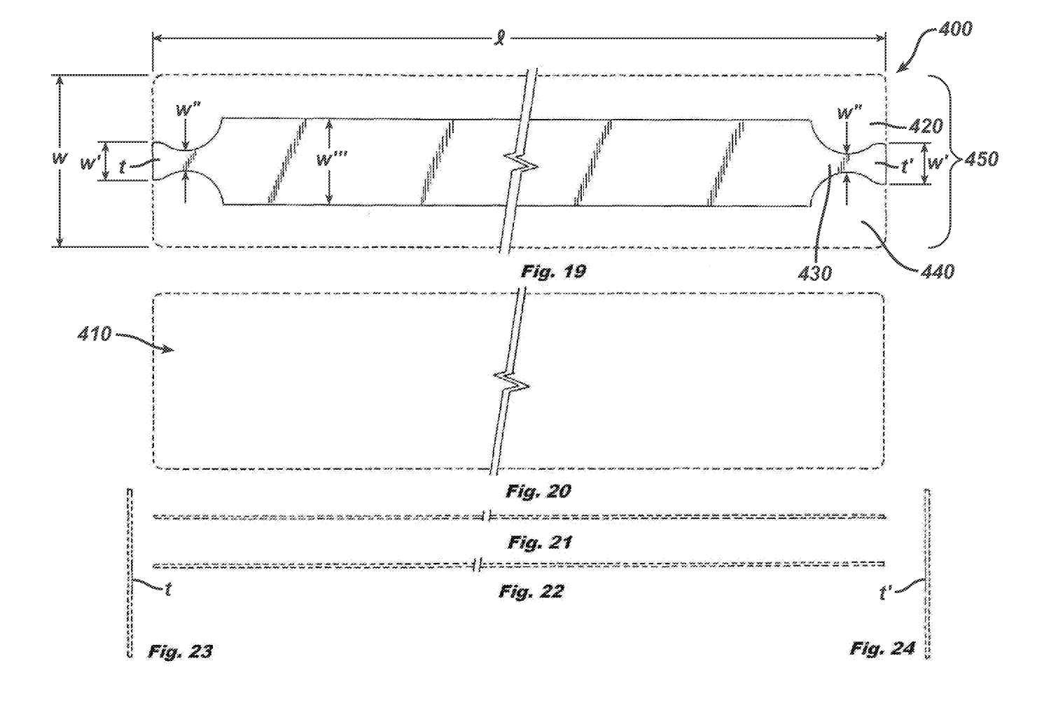

FIGS. 19-24 depict a wound closure device comprising a three-part release liner in accordance with another embodiment of the invention.

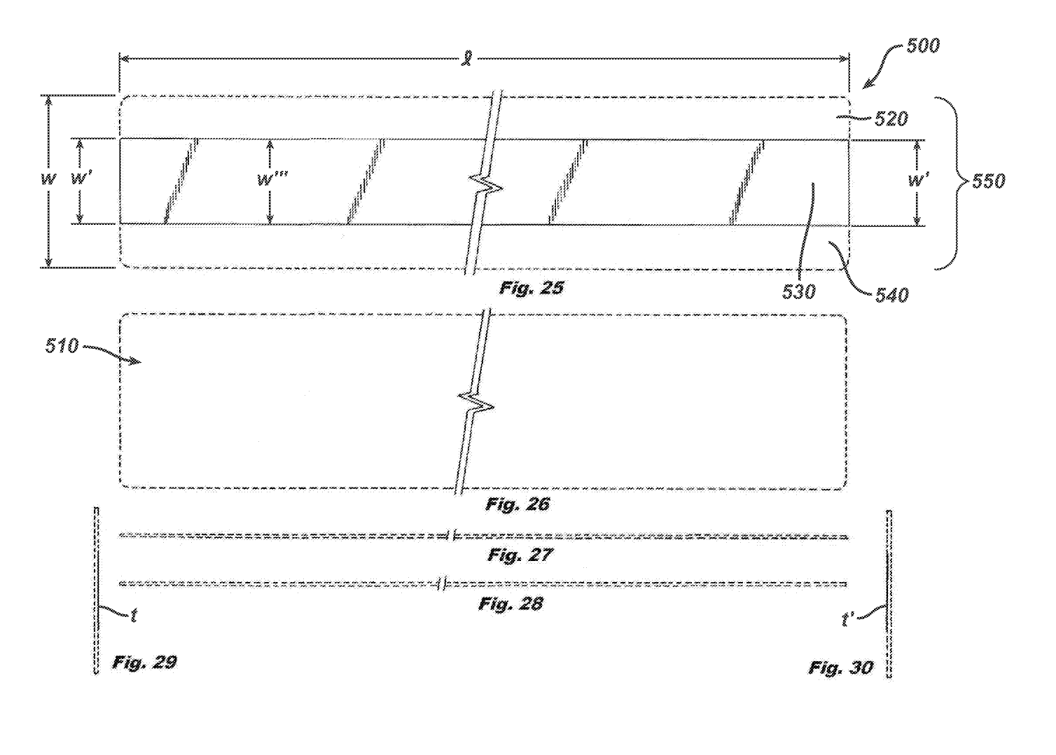

FIGS. 25-30 depict a wound closure device comprising a three-part release liner in accordance with a further embodiment of the invention. In this embodiment, central section 530 is of continuous width along the entire length l of device 500. Therefore, widths w′ of right and left ends of central section 530 are equal to width w′″ along the length l of central section 530.

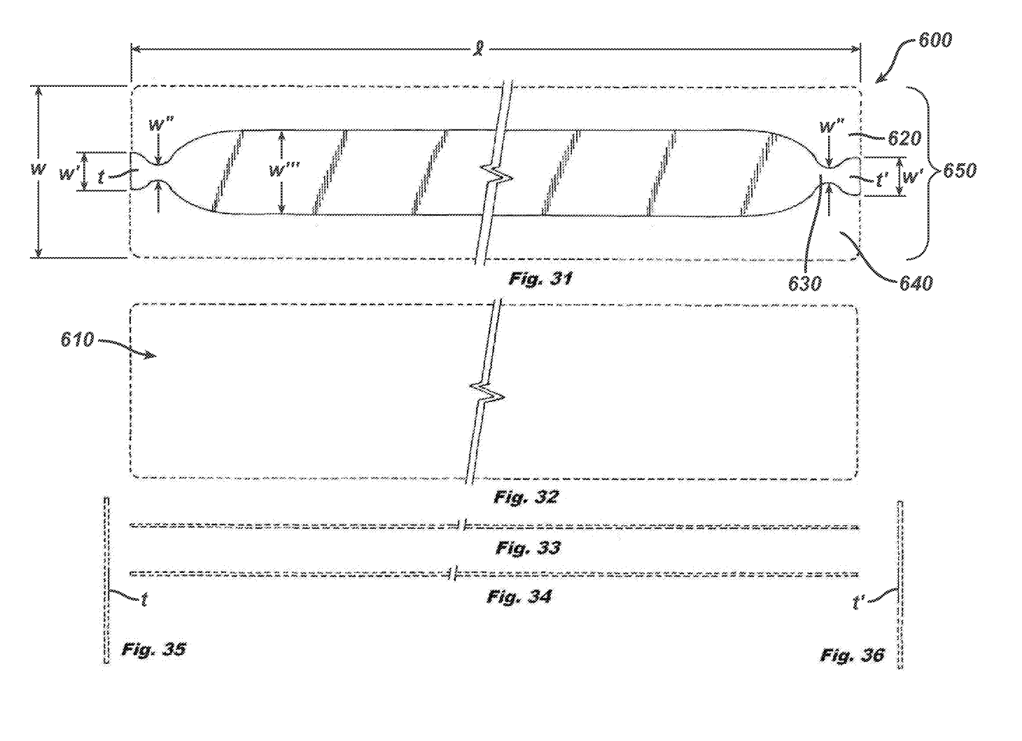

FIGS. 31-36 depict a wound closure device comprising a three-part release liner in accordance with another embodiment of the invention.

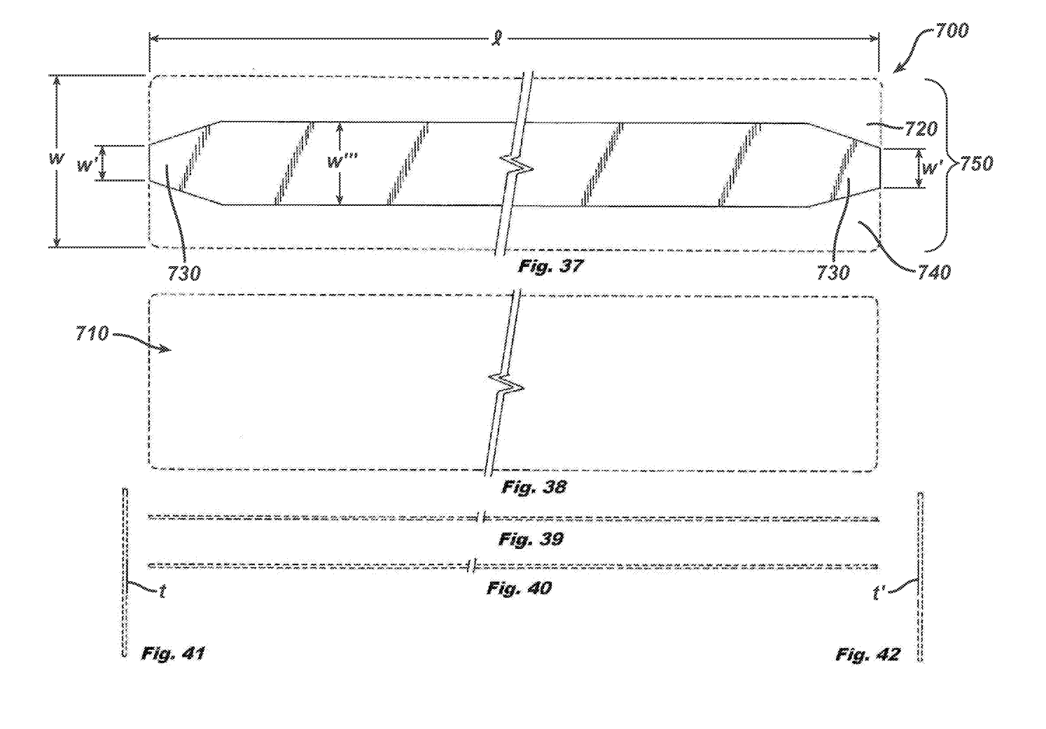

FIGS. 37-40 depict a wound closure device comprising a three-part release liner in accordance with a further embodiment of the invention. In this embodiment, central section 730 varies in width wherein central section 730 has width w′ at the left edge of central section 130 which increases to width w′″ before reducing to width w′ at the right edge of central section 130.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A wound treatment device comprising a three-part release liner assembly, the release liner assembly comprising a first frame, a central section, and a second frame.

2. The device of claim 1 wherein central section of the release liner assembly comprises a length and a width, wherein the width varies along the length of the central section and has a left edge and a right edge and wherein along the length of the central section, the width of the central section has a first width at the left edge which decreases to a second width, which further increases to a third width, which further decreases to a fourth width and which further increases to a fifth width at the right edge.

3. The device of claim 1 wherein the central section of the release liner assembly comprises a length and a width, wherein the width varies along the length of the central section and has a left edge and a right edge and wherein along the length of the central section, the width of the central section has a first width at the left edge that increases to a second width for a length and then decreases to a third width at the right edge.

4. The device of claim 1 wherein the central section of the release liner assembly comprises a length and a width, wherein the central section and has a left edge and a right edge and wherein along the length of the central section, the width of the central section has a width at the left edge wherein the width not vary along the length of central section and is maintained up to the width at the right edge.

5. A wound treatment device comprising a wound treatment component, the wound treatment component comprising a wound facing side and a top side, the wound facing side detachably adhered to a three-part release liner assembly, the release liner assembly comprising a first frame, a central section, and a second frame.

6. The wound treatment device of claim 5, wherein the central section comprises a length and a width, wherein the width varies along the length of the central section and has a left edge and a right edge and wherein along the length of the central section, the width of the central section has a first width at the left edge which decreases to a second width, which further increases to a third width, which further decreases to a fourth width and which further increases to a fifth width at the right edge.

7. The wound treatment device of claim 5, wherein the central section of the release liner assembly comprises a length and a width, wherein the width varies along the length of the central section and has a left edge and a right edge and wherein along the length of the central section, the width of the central section has a first width at the left edge that increases to a second width for a length and then decreases to a third width at the right edge.

8. The wound treatment device of claim 5, wherein the central section of the release liner assembly comprises a length and a width, wherein the central section and has a left edge and a right edge and wherein along the length of the central section, the width of the central section has a width at the left edge wherein the width not vary along the length of central section and is maintained up to the
